(19) United States
(12) Patent Application Publication
(10) Pub. No.: US 2014/0196722 A1
(43) Pub. Date: Jul. 17, 2014

(54) TRACHEAL TUBE SYSTEM FOR ENHANCED PATIENT COMFORT

(71) Applicant: COVIDIEN LP, Mansfield, MA (US)

(72) Inventors: Kamlesh Sethiya, Athlone (IR); Emmet Bolger, Athlone (IR)

(73) Assignee: COVIDIEN LP, Mansfield, MA (US)

(21) Appl. No.: 13/742,186

(22) Filed: Jan. 15, 2013

(51) Int. Cl. A61M 16/04; A61M 16/00

(52) U.S. Cl. CPC A61M 16/0497; A61M 16/0434; A61M 16/0057
USPC 128/207.17; 128/207.14; 29/428

(57) ABSTRACT

A tracheal tube system includes a tracheal tube assembly having a cannula configured to be positioned in a patient airway, and a connector coupled to the proximal end of the cannula. The tracheal tube assembly further includes a flange member secured about the cannula. The flange member includes an interior face configured to be disposed onto a patient neck when in use and a first plurality of protrusions disposed on top of the interior face, wherein the cannula and the connector form a contiguous passageway for exchanging fluid with the patient airway in operation.

### BACKGROUND

The present disclosure relates generally to the field of tracheal tubes and, more particularly, to a tracheal tube assembly that enhances patient comfort.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For certain applications, particularly when longer-term intubation is anticipated, tracheostomy tubes may be preferred. Tracheostomy tubes are typically inserted through an incision made in the neck of the patient and into the trachea. A resulting stoma is formed between the tracheal rings below the vocal chords. The tracheostomy tube is then inserted through the opening.

Such tubes may include an inner cannula and an outer cannula, with the inner cannula, may be disposed inside the outer cannula and used as a conduit for liquids or gas or medicine incoming and outgoing into the patient's lungs. The inner cannula may be removed for cleaning and for disposal of secretions while leaving the outer cannula in place, thus maintaining a desired placement of the tracheostomy tube. A connector is typically provided at an upper or proximal end where the tube exits the patient airway, suitable for coupling the ventilator with the inner cannula. A set of flanges or wings are disposed around the outer cannula and used to securely couple the tracheostomy tube to the patient neck. Standard flanges have been developed various sizes to more comfortably secure the tracheostomy tube against the patient neck. For example, flanges may come in pediatric, neonatal and adult sizes designed to more easily accommodate a varying number of necks anatomies. The flanges may include openings through which for example, a strap such as a cloth or plastic strap maybe used to secure the flange to the patient. One difficulty that arises in the use of tracheal tubes, and tracheostomy tubes in particular, is in securing the tracheostomy tube more comfortably to the patient particularly during long term use. This may lead to difficulties of the tracheostomy tube becoming decoupled from the patient neck or difficulties in patient comfort. For example, during long term use the tracheal tube may move laterally from the neck and become dislodged. Additionally the design of typical tracheal tubes may not be as comfortable as desired.

There is a need, therefore, for improved tracheal tubes, and particularly for improved flanges and/or straps of tracheostomy tubes. It would be desirable to provide a tube and tube flange that allows for greater facility in securing the tracheal tube to the patient and that also provides for greater facility during use, for example, by enhancing patient comfort.

### BRIEF DESCRIPTION

This disclosure provides a novel tracheal tube designed to respond to such needs. The tube allows for a set of flanges or wings having a plurality of protrusions on a face of the flange facing or contacting a patient's neck. In certain embodiments, these protrusions may be manufactured out of a material different than the material used to manufacture a main flange body. For example, these protrusions may be manufactured of a material softer than the material used to manufacture the flange body. In one example, such material may include a polyurethane foam having a durometer hardness of approximately between shore OO and shore D, for example between durometer 1 and durometer 70. The soft protrusions may contact the patient neck and aid in securing the flange to the patient neck, for example, by providing for enhanced friction. Additionally, the protrusions may enable for the flow of air under the flange, thus providing for enhanced comfort and a flow suitable for minimizing patient sweat and/or for enabling the evaporation of sweat. Accordingly, the tracheostomy tube may be more securely attached to the patient neck, and additionally provide for increased comfort.

Additionally, the protrusion may be disposed on the strap used to attach the tracheal tube to the patient's neck. For example, the protrusions may be disposed on the patient-facing side of the strap, and used to more comfortably contact the neck when the strap is in use. By providing for soft protrusions on the flange and/or the neck strap, the techniques described herein may enable a more comfortable tracheal tube assembly suitable for longer wear and use.

Thus, in accordance with a first aspect, a tracheal tube system is provided. The tracheal tube system includes a tracheal tube assembly having a cannula configured to be positioned in a patient airway, and a connector coupled to the proximal end of the cannula. The tracheal tube assembly further includes a flange member secured about the cannula. The flange member includes an interior face configured to be disposed onto a patient neck when in use and a first plurality of protrusions disposed on top of the interior face, wherein the cannula and the connector form a contiguous passageway for exchanging fluid with the patient airway in operation.

In accordance with another aspect, a tracheal tube assembly is provided. The tracheal tube assembly includes an outer cannula configured to be positioned in a patient airway and an inner cannula configured to be disposed inside the outer cannula. The tracheal tube assembly additionally includes a connector coupled to the proximal end of the outer cannula and a flange member secured about the outer cannula. The flange member includes an interior face configured to be disposed onto a patient neck when in use and a first plurality of protrusions disposed on the interior face, wherein the inner cannula and the connector form a contiguous passageway for exchanging fluid with the patient airway in operation.

Also disclosed herein is a method of manufacturing a tracheostomy system. The method includes manufacturing a cannula configured to be positioned in a patient airway and manufacturing a connector coupled to the proximal end of the cannula. The method additionally includes manufacturing a flange member secured about the cannula comprising an interior face configured to be disposed onto a patient neck when in use. The method further includes manufacturing a first plurality of protrusions on top of the interior face, wherein the cannula and the connector form a contiguous passageway for exchanging fluid with the patient airway in operation.

### BRIEF DESCRIPTION OF THE DRAWINGS

Various aspects of the disclosed techniques may become apparent upon reading the following detailed description and upon reference to the drawings in which:

FIG. 1 is a perspective view of an exemplary tracheal tube assembly, in accordance with aspects of present techniques;

FIG. 2 is a frontal view of the exemplary tracheal tube flange of FIG. 1 showing a plurality of protrusions;

FIG. 3 is a top view of the exemplary tracheal flange of FIG. 2 showing the plurality of protrusions;

FIG. 4 is a detail view taken through arc 4-4 of FIG. 3 showing certain components of the flange and protrusions;

FIG. 5 another embodiment of a top view of taken through arc 4-4 of FIG. 3 showing certain components of the flange and protrusions;

FIG. 6 is a detail view of a single protrusion shown in FIG. 5 above, showing certain manufacturing techniques useful in attaching the protrusion to the flange;

FIG. 7 is a view of a plurality of shapes that may be useful for the protrusions shown in FIGS. 3 to 6;

FIG. 8 is a detail view taken through arc 8-8 of FIG. 1 of a strap including a plurality of protrusions;

FIG. 9 is a frontal view showing distances useful in disposing the protrusions;

FIG. 10 is a frontal view showing other distances useful in disposing the protrusions;

FIG. 11 is a frontal view showing protrusions having different sizes; and

FIG. 12 is an embodiment of a process useful in manufacturing the tracheal tube assembly of FIG. 1.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

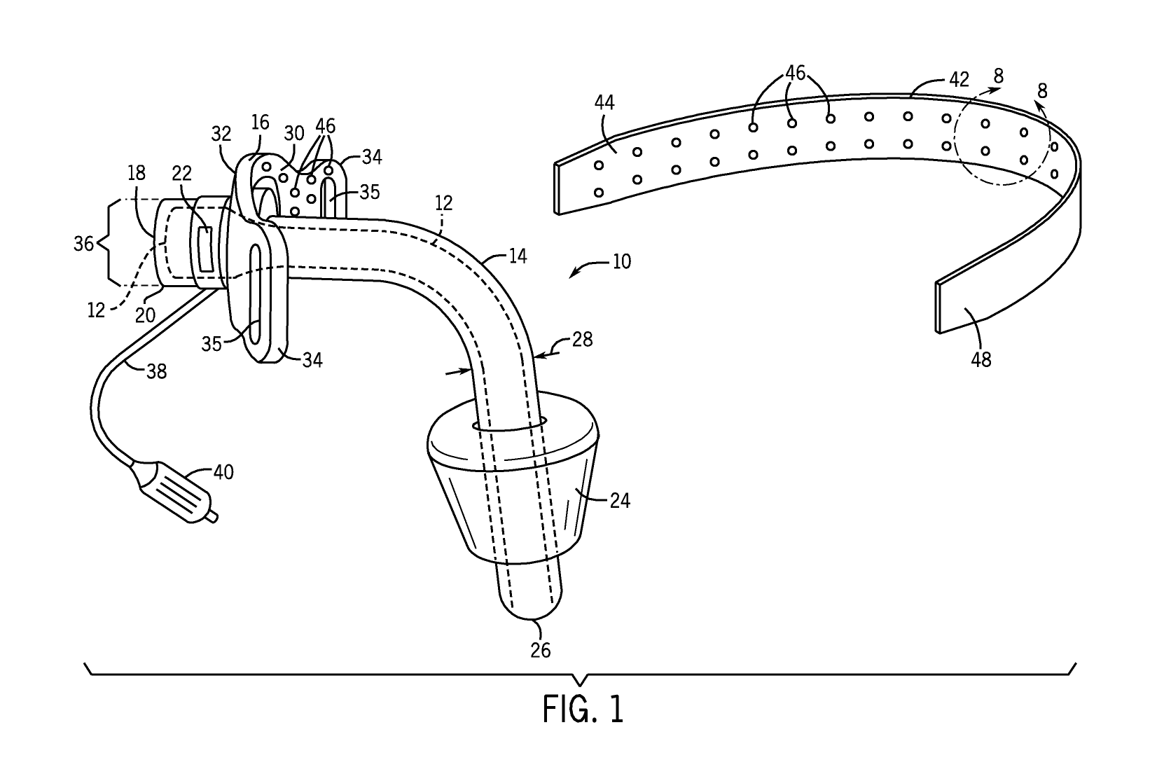

A tracheal tube according to a preferred embodiment is illustrated in FIG. 1. While, the tracheal tube system 10 represented in the figures is a tracheostomy tube, aspects of this disclosure could be applied to other tracheal tube structures, such as endotracheal tubes. The application to a tracheostomy tube is apt, however, insomuch as such tubes tend to be worn for longer periods of time, and thus may include a removable and/or disposable inner cannula 12 shown disposed inside of an outer cannula 14, useful in maintaining a clean ventilation circuit.

The outer cannula 14 is illustrated extending both distally as well as proximally from a flange member 16. The inner cannula 12 may be introduced through an opening 18 of an end connector 20 and disposed inside of the outer cannula 14. During intubation, a tracheal tube assembly 22 including the inner and outer cannulae 12, 14 is placed through an opening formed in the neck and trachea of a patient, and extending into the patient airway. The tube assembly 22 embodiment illustrated in the figures includes a sealing cuff 24, although in practice a wide range of tube designs may be used, including tubes having no cuffs or tubes having multiple cuffs around the outer cannula 14. The inner cannula 12 in the illustrated embodiment forms a conduit from which liquids or gases, including medications, may enter through the proximal opening 18 an exit through a distal opening 26. The cannula has an outer dimension 28 allowing it to fit easily through an incision made in the neck and trachea of the patient. In practice, a range of such tubes may be provided to accommodate the different contours and sizes of patients and patient airways. Such tube families may include tubes designed for neonatal and pediatric patients as well as for adults. By way of example only, outer dimension 28 of the tube 20 may range from 4 mm to 16 mm.

In one embodiment, the outer cannula 14 enters the flange member 16 along a lower face 30 and protrudes through an upper face 32 of the flange member 16. When in use, the face 30 will generally be positioned against the neck of a patient, with the cannula extending through an opening formed in the neck and trachea. A pair of side wings or flanges 34 extend laterally and serve to allow a strap or retaining member 36 to hold the tube assembly in place on the patient. In the illustrated embodiment, apertures 37 are formed in each side flange 34 to allow the passage of such a retaining device. In many applications, the flange member 16 may be taped or sutured in place as well.

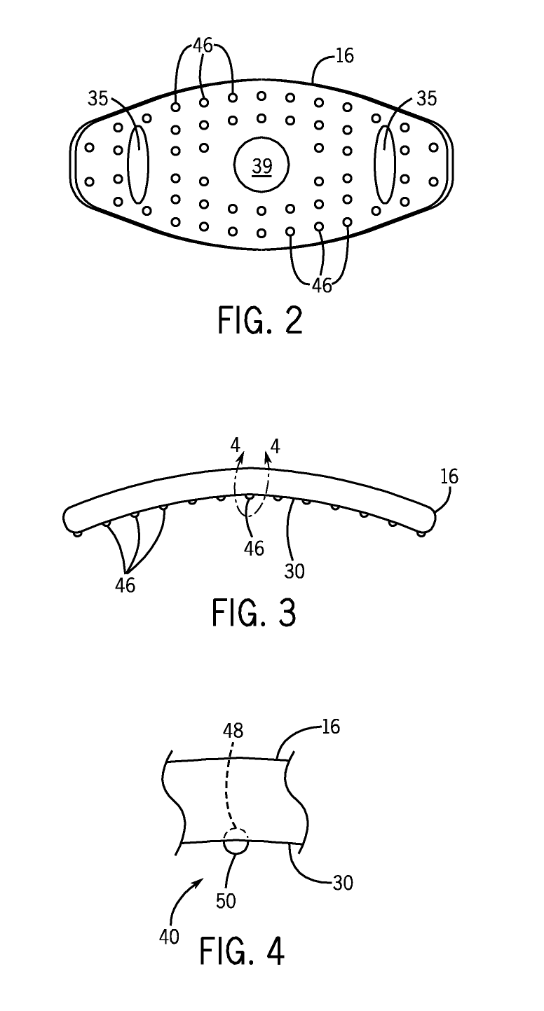

The end connector 20 is formed in accordance with industry standards to permit and facilitate connection to ventilating equipment (not shown). By way of example, standard outer dimensions may be provided as indicated at reference numeral 38 that allow a mating connector piece to be secured on the connector shown. By way of example, a presently contemplated standard dimension 38 accommodates a 15 mm connector, although other sizes and connector styles may be used. In use, then, air or other gas may be supplied through the connector and the inner cannula 12, and gases may be extracted from the patient. For example, the tube system 10 may be inserted into the patient's airways, and the cuff 24 may then be inflated through an inflation lumen 39. A pilot balloon 40 may then indicate that air is in the cuff 24, thus sealing the patient's airway. Once the tracheal tube is positioned and secured, a ventilator may be coupled to the end connector 20, as described in more detail below with respect to FIG. 2.

Also shown are a plurality of protrusions 46 disposed on top of the face 30 of the flanges 34. The protrusions 46 may enhance patient comfort and additionally provide for a more secure attachment of the tracheal tube assembly 22 to the patient. For example, the protrusions 46 may include a soft polymer material that more comfortably and comformably adapts to the patient neck and that may additionally be used provide an enhanced friction between the tracheal face 30 and the patient neck. For example, the protrusions 46 may deform slightly when contact or abutment pressure is applied but may still provide for a flow of air between the neck and the flanges 34.

Additionally or alternatively, the neck strap 36 may also be used to secure the tracheal tube assembly 22 to the neck. The next strap 36 may include a patient-facing side or face 48 having the plurality of protrusions 46. In use, strap 36 may be placed against the patient neck so that the face 48 contacts the neck. Strap ends 47 may then be threaded through the openings 37 used to secure the tracheal tube assembly 22 to the patient. As mentioned above, in one embodiment, the protrusions 46 may be manufactured of a soft material having a durometer hardness of between 1 and 70, such as soft polymer foam. When disposed against the patient, the protrusions 46 may more comformably fit the neck and enable a more comfortable and secure attachment of the strap 36 to the patient.

Turning now to FIG. 2, a frontal view depicting the face 30 of the flange member 16 is shown. More specifically, the flange member 16 is depicted in the x-y plane of an axis 51. In one embodiment, the face 30 may include a plurality of the protrusions 46 disposed throughout the entirety of the face 30. In another embodiment, the protrusions 46 may be disposed in selected sections of the face 30, such as only near the openings 35, near opening 49, between the opening 49 and the openings 35, or in any desired location. The protrusions 46 may be manufactured by disposing a plurality of spheres, partial spheres, or other shapes, onto the inner face 30, as described in more detail below with respect to FIG. 7.

In use, the openings 35 are suitable for providing insertion of the ends 47 of the neck strap 36, and the securing the neck strap 36 to the patient neck. As mentioned above the opening 49 may be used to insert the outer cannula 14 so as to dispose the flange member 16 about the outer cannula 14. The inner cannula 12 may then be disposed inside of the outer cannula 14. The resulting tube assembly 22 having the inner and outer cannulae 12, 14 may then be inserted into the patient trachea, and coupled to a medical device, such as a ventilator. The ventilator may then provide gases and other medicine into the patient and may aid in ventilating the patient when in use.

In one example, the plurality of protrusions 46 and the flange 16 may be manufactured out of a material such as polyvinylchloride, a polyurethane, thermoplastic elastomers, a polycarbonate plastic, silicon, an acrylonitrile butadiene styrene (ABS), or a polyvinyl chloride (PVC), rubber, neoprene, or combination thereof. In certain embodiments, the flange 16 may be manufactured out of a different material than the protrusions 46. However, in another embodiment, the protrusions 46 and the flange member 16 may be both manufactured of the same material. In one embodiment, the flange 16 may be manufactured out of a first material having a harder durometer measure when compared to a second material used in manufacturing the plurality of protrusions 46. Accordingly a more rigid and stable flange may be provided, having a softer underside 30 with the inclusion of the plurality of protrusions 46. In other embodiments, the first durometer hardness may be less than or equal to the second durometer hardness. Additional or alternative to providing enhanced comfort, the protrusions 46 may enable certain flow, such as flow of air, under the patient neck. According, the protrusions 46 may provide for a more comfortable cooling flow under the flange 16, especially desirable in situations when a patient may be encountering a fever or other medical issue leading to undesired thermal conditions.

FIG. 3 is a top view of the flange 16 showing the plurality of protrusions 46 disposed on the x-z plane of the axis 51. As illustrated, the protrusions 46 may extend a certain distance or height H1 away from the face 30 of the flange 16. The height H1 may be between 0.01 mm and 10 mm. By extending to the height H1, the protrusions 46 may first contact the patient anatomy, instead of the face 30. Additionally, the height H1 may enable the protrusions 46 to provide for a flow of gases between the flange 16 and the patient neck, thus allowing, for example, for improved evaporation of patient sweat and additionally providing for increase air flow through the neck region under the tracheostomy flange 16. By enabling an improvement in the evaporation of sweat and providing for the flow of air under the flange 16, the protrusions 46 may enhance patient comfort and increase the amount of time that the tracheostomy tube system 10 may be worn by the patient. The protrusions 46 may include certain shapes, such as spheres or partial spheres, as shown in more detail with respect to FIG. 4 below.

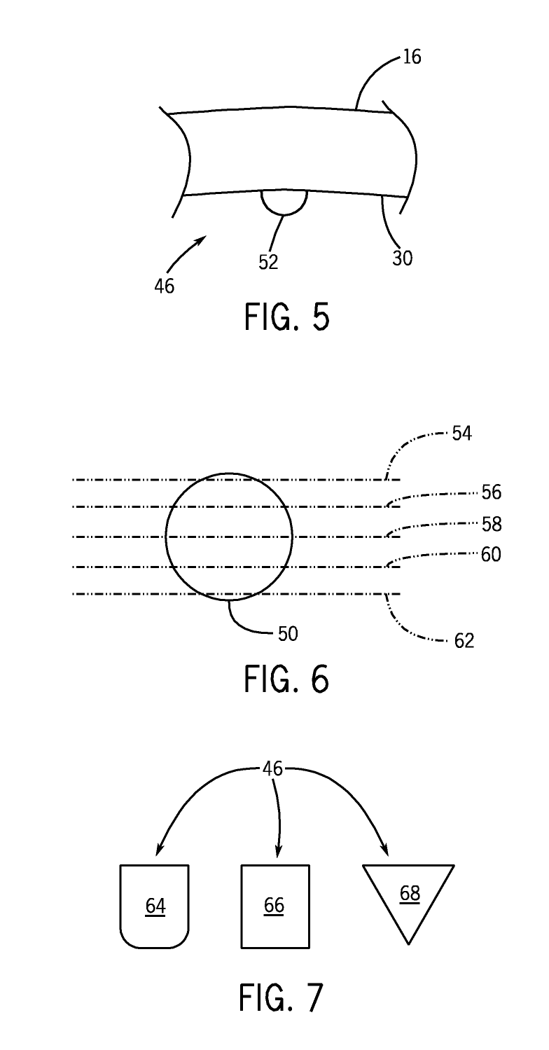

FIG. 4 is a view taken through arc 4-4 of FIG. 3 above, showing a detail view of the protrusions 46 including a semispherical shape 50 disposed onto the face 30 of the flange 16. In other embodiments, the protrusions 46 may be disposed on one or more layers of other material (e.g., soft material such as neoprene, fabric, and so on) and the one or more layers may then be disposed onto the face 30. The protrusions 46 may include a size having a width of between 0.01 mm to 15 mm, a depth of between 0.01 mm to 15 mm, and the height H1. As mentioned above, by providing for the protrusions 46, the techniques described herein may enable a more comfortable and secure tracheostomy tube system 10. In one example, the protrusions 46 may be manufactured by molding or overmolding the shape 50 on top of the face 30. In another example, the protrusions 46 may be manufactured as having the shape 50 by computer numerically controlled (CNC) techniques, milling techniques, and so on. In yet another embodiment, the protrusions 46 may be manufactured separate from the flange 16 and then adhered to the flange 16, for example, by using thermal bonding, adhesives, and similar techniques. In certain embodiments, the protrusions 46 may manufactured as having full spherical shapes, and then adhered into dimples or cavities included in the face 30, described in more detail below with respect to FIG. 5.

FIG. 5 is an embodiment of a top view also taken through arc 4-4 of FIG. 3 above, showing a detail view of the protrusion 46 as including a full spherical shape 52 disposed on top of the face 30 of the flange 16. In the depicted embodiment, the protrusions 46 may be “micro balls” having the shape 52, and may be manufactured out of the previously aforementioned materials (e.g., polyvinylchloride, a polyurethane, thermoplastic elastomers, a polycarbonate plastic, silicon, an acrylonitrile butadiene styrene (ABS), or a polyvinyl chloride (PVC), rubber, neoprene, or combination thereof). In one embodiment, the face 30 may include dimples or cavities 53 having a shape matching the contours of the protrusion 46. Accordingly, the protrusion 46 may be more securely placed onto the face 30. Each protrusion 46 may be molded, overmolded, or otherwise adhered onto a respective cavity 53. The protrusions 46 may be disposed at varying depts. into the face 30 of the flange 16, as described in more detail below with respect to FIG. 6.

FIG. 6 is a view along the x-z plane of the axis 51 of the protrusion 46 showing different depths of assertion of the protrusion 46 into the face 30 of the flange 16. Indeed, the protrusions 46 may be disposed at any number of depths and extend outwardly from the face 30 at the extension height H1 previously described with respect to FIG. 3 above. In the depicted embodiment, a first insertion depth marked by dashed line 54 may enable a protrusion 46 extending from the flanged face 30 to extend to a height almost approximately equal to a diameter or the full height of the protrusion 46. In this embodiment, the insertion line 54 may provide for the greatest height of extension, thus increasing the amount of sweat evaporation and the air flow under the flange 30. Other insertion lines 56, 58, 60, and 62, may provide for lesser heights H1 when compared to the insertion line 54. For example, insertion line 56 may result in the protrusion 46 extending at a height smaller then the height provided by the insertion line 54. Similarly, insertion line 58 may result in a height smaller when compared to the height provided by assertion line 56. Likewise, insertion line 60 may provide for a smaller extension height when compared to the height provided by insertion line 58. Additionally, insertion line 62 may provide for a smaller height when compared to the height provided by insertion line 60. By providing for any number of insertion lines, such as the lines 54, 56, 58, 60 and 62, any number of heights H1 may be achieved. Additionally, the protrusions 46 may be provided in a variety of shapes, as described in more detail with respect to FIG. 7 below.

FIG. 7 is a view of shapes 64, 66, and 68 that may be included in the protrusions 46. More specifically, the figure depicts the shapes 64, 66, and 68 along the x-z plane of the axis 51. The shape 64 may be a conical shape having a rounded face 65. The conical shape 64 may be more easily inserted into the face 30 of the flange 16, while the rounded end 65 may provide for a more comfortable contact against the patient neck. Likewise, the rectangular shape 66 may be provided having a square contact face 67. The rectangular shape 66 may enhance contact friction by providing for a larger contact face 67, and may also provide, for example, right angles α, β useful in abutting the patient skin at right angles, thus increasing the attachment of the flange and/or strap to the patient neck.

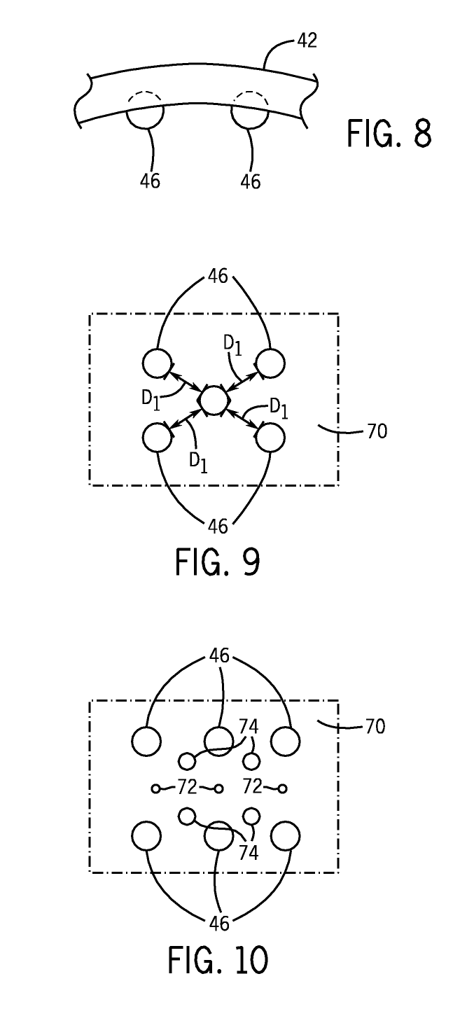

In certain embodiments, such as embodiments where the patient may be undergoing more movement, a shape 68 having a pointed end 69 may be provided. For example, shape 68 may be particularly useful in emergency situations where the patient is going to be moved in a variety of vehicles such as an ambulance, a helicopter, and so on. The pointed end 67 may provide for a more secure contact point when placed against the patient's neck. Other shapes may be provided, including hexagonal shapes octagonal shapes, curved shapes, and so on. It is to be understood, that, in addition to or alternative to the protrusions 46 being disposed onto the flange 16, the protrusions 46 may be disposed onto the neck strap 42 as illustrated in FIG. 8.

FIG. 8 is a top view of the neck strap 36 including the protrusions 46. In the depicted embodiment, the neck strap 36 may be manufactured out of a fabric (e.g., cloth fabric, vinyl fabric, polyester fabric, polyvinylchloride, a polyurethane, thermoplastic elastomers, a polycarbonate plastic, silicon, an acrylonitrile butadiene styrene (ABS), or a polyvinyl chloride (PVC), rubber, neoprene, or combination thereof. In one embodiment, the protrusions 46 may include a first durometer hardness lesser than a second durometer hardness included in the strap 36. In other embodiments, the first durometer hardness may be equal to or greater than the second durometer hardness.

As mentioned above with respect to the manufacturing of the protrusions 46, the protrusions 46 may adhered, molded, or overmolded onto the strap 36. By disposing the protrusions 46 onto the neck strap 36, the protrusions 46 may also provide for enhanced comfort and security when the neck strap 36 is attached to the patient neck. For example, the protrusions 46 may provide for enhanced friction enabling the neck strap 36 to be more securely attached to the neck even during circumstances when the patient is moved. Accordingly, a force used to secure the neck strap 36 to the flange 16 through the opening 35 may be less of a force when compared to the attachment force when the protrusions 46 are not used. When placed onto the patient-facing side 48 of the strap 36, the protrusions 46 may extend at a distance or height H2 away from the face 48. In one embodiment, the H2 may be the approximately the same as the height H1. In other embodiments, H2 may be smaller or greater than H1. By using the protrusions 46, the patient may be more conformably, securely, and snuggly fit to the neck strap 36. Further, the protrusions 46 may be disposed at different distances or the same distance with respect to each other, as shown in more detail with respect FIG. 9 below.

FIG. 9 is a frontal view of the protrusions 46 where each protrusion 46 is disposed at a uniform distance D1 with respect to its closest neighbor. More specifically, the protrusions 46 are shown disposed onto a substrate 70 along the x-y plane of the axis 51, such as the substrate 70 found in the face 30 of the flange 16 and/or in the face 48 of the neck strap 42, at a uniform distance D1. That is, the distance D1 is same distance from a protrusion 46 to each near neighbor. Such equidistant positioning of the protrusions 46 may enable for a more uniform contact or abutment force. It is to be noted, that, in other embodiments, the positioning of the protrusions 46 with respect to each other may be not equidistant but may be random or may have different positioning closer or further away with respect to one other, as described in more detail with respect to FIG. 10 below.

FIG. 10 depicts a frontal view on the x-y plane of the axis 51 of the protrusions 46 disposed at different distances D2, D3, and D4, from near neighbors. That is, the distance D2 may be different from the distance D3 which may be different from the distance D4. In certain embodiments, the distance D2, D3, and D4, may be selected to be between range between 0.1 mm and 15 mm. By disposing the protrusions 46 at differing sizes D2, D3, and D4, the techniques described herein may provide for an enhanced gripping area on the substrate 70 such that the protrusions 46 may contact the patient neck at different locations.

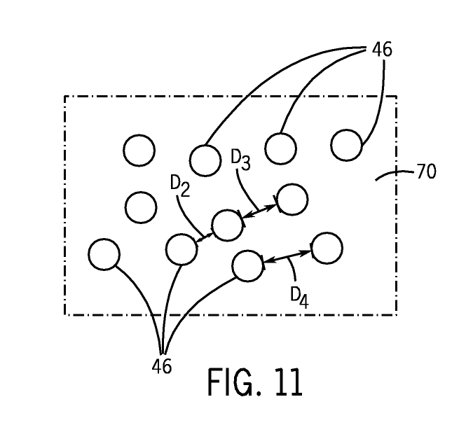

Turning to FIG. 11, the figure is a frontal view on the x-y plane of the axis 51 showing the protrusions 46 having different sizes 72, 74, and 75. In the depicted embodiment, the protrusions 72 are smaller protrusions 72 when compared to protrusions 74 and 75. By disposing protrusions 46 of different sizes, such as the sizes 72, 74, and 76, the techniques described herein may provide for a more adhesive friction force between the substrate 70 and the patient neck. For example, certain and anatomical features, such as moles or bumps on the neck, may abut certain of the protrusions with a more uniform force compared to protrusions having all the same size. Accordingly, the multiple size protrusions 70, 72, 74, and 75, may more easily fit the variety of patient anatomies and may provide for a more secure coupling of the substrate 70 to the patient neck. As mentioned before, the protrusions may have a width of between 0.01 mm to 15 mm, a depth of between 0.01 mm to 15 mm, and a height of between 0.01 mm to 10 mm.

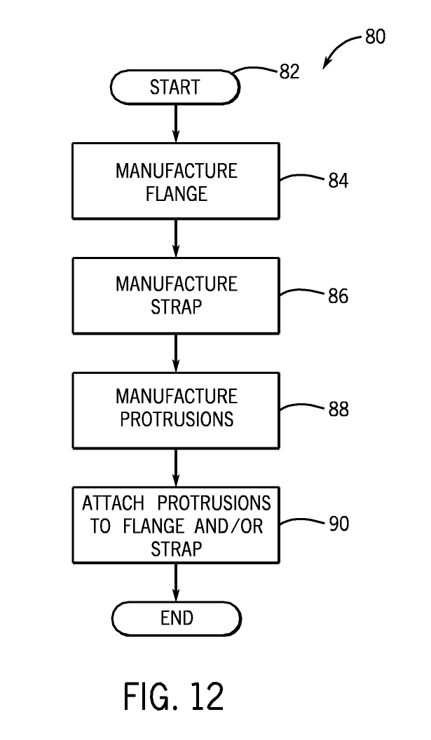

FIG. 12 is an embodiment of a process 80 that may be used to manufacture the tracheal tube assembly of FIG. 1. The process 80 may be implemented as computer instructions or code stored in non-transitory computer readable medium, such as a memory of a controller used in manufacturing. In the depicted embodiment, the process 80 may begin at block 82. The process 80 may then manufacture the flanges 34 (block 84). The flanges may be manufactured out of polyvinylchloride, a polyurethane, thermoplastic elastomers, a polycarbonate plastic, silicon, an ABS, a PVC, rubber, neoprene, or combination thereof. The manufacturing (block 84) may include, for example, molding or overmolding the flanges, or CNC milling the flanges.

The process 80 may then manufacture (block 86) the strap 42 in similar fashion. For example, the strap may be molded, overmolded, and/or CNC milled out of polyurethane, thermoplastic elastomers, polycarbonate plastic, silicon, ABS, PVC, rubber, neoprene, or combination thereof. The process 80 may then manufacture the protrusions 46 (block 88). In one embodiment, the protrusions 46 may be manufactured at a lower hardness than the strap 42 and/or flanges 34. In another embodiment, the protrusions 46 may be manufactured at the same hardness of the strap 42 and/or flanges 34. In yet another embodiment, the protrusions 46 may be manufactured at a higher hardness than the strap 42 and/or flanges 34. By providing for different hardness levels of the protrusions 46 compared to the strap 42 and/or flanges 34, the techniques described herein can provide different levels of patient comfort, rigidity of the protrusions 46, and friction forces provided by contact of the neck with the protrusions 46.

The process 80 may then attach the protrusions 46 to the strap 42 and/or flanges 34 (block 90). For example, the protrusions 46 may be molded or overmolded onto the strap 42 and/or flanges 34, bonded onto the strap 42 and/or flanges 34, and the like.

What is claimed is:

1. A tracheal tube system comprising:
a tracheal tube assembly comprising:
a cannula configured to be positioned in a patient airway;
a connector coupled to the proximal end of the cannula;
a flange member secured about the cannula comprising:
an interior face configured to be disposed onto a patient neck when in use; and
a first plurality of protrusions disposed on top of the interior face, wherein the cannula and the connector form a contiguous passageway for exchanging fluid with the patient airway in operation.

2. The system of claim 1, wherein at least one of the first plurality of protrusions comprises a hemispherical shape, a spherical shape, a triangular shape, a rectangular shape, or combination thereof.

3. The system of claim 1, wherein the interior face comprises a first durometer hardness measure and each of the first plurality of protrusions comprises a second durometer hardness measure different than the first durometer hardness measure.

4. The system of claim 1, wherein each of the first plurality of protrusions comprises a durometer hardness measure of approximately between shore OO and shore A.

5. The system of claim 1, wherein each of the first plurality of protrusions are separated at a distance of approximately between 0.01 mm to 10 mm from each other.

6. The system of claim 1, wherein the first plurality of protrusions are separated at approximately the same distance from each other.

7. The system of claim 7, wherein the flange member comprises a second plurality of protrusions disposed on the interior face, and the second plurality of protrusions are separated at different distances from each other.

8. The system of claim 1, wherein each of the first plurality of protrusions extends outwardly from the interior face at a height of approximately between 0.01 mm to 10 mm

9. The system of claim 1, comprising an inner cannula disposed inside the cannula, wherein the inner cannula and the connector form a contiguous passageway for exchanging fluid with the patient airway in operation.

10. The system of claim 1, comprising a strap having a second plurality of protrusions disposed on a patient-facing strap side, wherein the tracheal tube is attached to the patient by the strap when in the strap is in use.

11. The system of claim 10, wherein at least one of the second plurality of protrusions comprises a spherical shape, a triangular shape, a rectangular shape, or combination thereof.

12. The system of claim 10, wherein the patient-facing strap side comprises a first durometer hardness measure and each of the second plurality of protrusions comprises a second durometer hardness measure different than the first durometer hardness measure.

13. A tracheal tube assembly comprising:
an outer cannula configured to be positioned in a patient airway;
an inner cannula configured to be disposed inside the outer cannula;
a connector coupled to the proximal end of the outer cannula;
a flange member secured about the outer cannula comprising:
an interior face configured to be disposed onto a patient neck when in use; and
a first plurality of protrusions disposed on the interior face, wherein the inner cannula and the connector form a contiguous passageway for exchanging fluid with the patient airway in operation.

14. The assembly of claim 13, wherein at least one of the first plurality of protrusions comprises a hemispherical shape, a partial hemispherical shape, a triangular shape, a rectangular shape, or combination thereof.

15. The assembly of claim 13, wherein the interior face comprises a first durometer hardness measure and each of the first plurality of protrusions comprises a second durometer hardness measure different than the first durometer hardness measure.

16. The assembly of claim 15, wherein the flange comprises at least two openings, and wherein a strap having a second plurality of protrusions disposed on a patient-facing side of the strap is configured to attach the patient neck to the flange by using the at least two openings.

17. A method of manufacturing a tracheostomy system comprising:
manufacturing a cannula configured to be positioned in a patient airway;
manufacturing a connector coupled to the proximal end of the cannula;
manufacturing a flange member secured about the cannula comprising an interior face configured to be disposed onto a patient neck when in use; and
manufacturing a first plurality of protrusions on top of the interior face, wherein the cannula and the connector form a contiguous passageway for exchanging fluid with the patient airway in operation.

18. The method of claim 17, wherein the manufacturing the first plurality of protrusions on top of the interior face comprises making the first plurality of protrusions separate from the flange member and then disposing the first plurality of protrusions on top of the interior face.

19. The method of claim 17, comprising manufacturing a strap having a second plurality of protrusions disposed on a patient-facing strap side, wherein the tracheal tube is attached to the patient neck by the strap when in the strap is in use.

20. The method of claim 17, wherein the interior face comprises a first durometer hardness measure and each of the first plurality of protrusions comprises a second durometer hardness measure different than the first durometer hardness measure.
